(19) United States
(12) Patent Application Publication
(10) Pub. No.: US 2023/0309921 A1
(43) Pub. Date: Oct. 5, 2023

(54) Window of Opportunity Skin Treatment Regimen and Composition for Preventing the Onset of or Treating Atopic Dermatitis

(71) Applicants: Johnson & Johnson Consumer Inc., Skillman, NJ (US); UNIVERSITY COLLEGE CORK - NATIONAL UNIVERSITY OF IRELAND, Cork (IE)

(72) Inventors: Thierry Oddos, Clamart (FR); Georgios Stamatas, Moulienaux (FR); Catherine Coret, Skillman, NJ (US); Jonathan Hourihane, Cork (IE); Alan D. Irvine, Cork (IE)

(21) Appl. No.: 18/124,977

(22) Filed: Mar. 22, 2023

Related U.S. Application Data
(60) Provisional application No. 63/327,052, filed on Apr. 4, 2022.

(51) Int. Cl. A61B 5/00; A61P 17/00

(52) U.S. Cl. CPC A61B 5/4848; A61P 17/00

(57) ABSTRACT

A window of opportunity skin treatment regimen and composition for preventing the onset of or treating atopic dermatitis is disclosed.

### CROSS REFERENCE TO RELATED APPLICATION

The present application claims the benefit of the earlier filing date of U.S. patent application 63/327,052, filed Apr. 4, 2022, the entirety of which application is hereby incorporated by reference herein as if fully set forth herein.

### FIELD OF THE INVENTION

The invention relates to a window of opportunity skin treatment regimen and composition that can be used to prevent the onset of or treat atopic dermatitis.

### BACKGROUND

The skin is a set of cells and macromolecules grouped together in the form of a resistant and flexible tissue which covers the entire body. It is made up of two joined layers, the epidermis and the dermis, and associated subcutaneous tissues.

The main function of the skin is to establish a protective barrier against environmental insults while allowing some exchanges between the internal and external environment. The barrier function is particularly important in limiting epidermal water loss. This function is provided chiefly by the corneal layer (stratum corneum), the uppermost layer of the epidermis, composed of flattened, anucleate cells called corneocytes. The watertightness of this “brick wall” is provided by an intercellular cement composed of specific lipids (cholesterol, cholesterol sulphate, free fatty acids and ceramides). The regenerative capacity of the epidermis is conferred by adult stem cells which allow regular replacement of the differentiated cells eliminated during keratinization. This process is particularly crucial for barrier function maturation and maintenance.

Adaptation to extrauterine life is a process which begins at birth and continues throughout the first year of life. The first months of postnatal life are a period of structural and functional reorganization of the skin allowing physiological adaptation to the extrauterine environment. For example, the immaturity of newborn skin is highlighted by the difference in the structure and molecular composition of the stratum corneum compared with that of adults. These are incomplete and thus continue to develop for at least the first 12 months after birth (Chiou et al., Skin Pharmacol Physiol, 17: 57-66, 2004; Nikolovski et al., J Invest Dermatol, 128: 1728-1736, 2008; Stamatas et al., Pediatr Dermatol, 27: 125-131, 2010; Telofski et al., Dermatol Res Pract, 2012: 198789, 2012). In addition, the results of two recent clinical studies (Fluhr et al., Br J Dermatol, 166(3): 483-90, 2012 and Fluhr et al., Br J Dermatol, 2014, 171(5): 978-86) suggest that infant skin presents a certain immaturity in its ability to capture water and regulate related mechanisms. Moreover, these studies have shown that the epidermal barrier organizes structurally from birth to 2 years of age and is therefore not completely mature during this period. This helps to explain the fragility of infants' and young children's skin and its susceptibility to chemical, physical and microbial attacks.

In addition, incomplete skin maturation can have significant clinical consequences. It is therefore important to allow the skin to be constructed and to develop properly and harmoniously, otherwise its functional and structural organization could be compromised. In this respect, it is crucial to preserve the barrier function and the renewal capacity of the epidermis.

Thus, the immaturity of the barrier and of the mechanisms regulating hydration in a baby's skin contributes to make it even more vulnerable to pathological situations such as atopic dermatitis.

Atopic dermatitis is one of the most common chronic diseases in the population. It is characterized by a set of clinical signs, the most important of which are pruritus and eczematous lesions, which may be acute, subacute or chronic. It almost always begins in infants or young children, while the barrier is structurally and functionally organizing itself. Atopic dermatitis usually begins at around three months of age, but sometimes in the first few weeks of life. It progresses in alternating relapse and remission phases. Depending on the child and the severity of the condition, it may last from several months to several years. A small percentage may persist into adulthood.

Atopic dermatitis is, first and foremost, a chronic inflammatory dermatological disease combining impairment of the skin barrier and skin inflammation. In a first sensitization phase, the skin barrier defect allows allergens to penetrate through the skin. Allergens that penetrate the upper layers of the epidermis are processed (internalized) by epidermal Langerhans cells and dermal dendritic cells. Langerhans cells are antigen-presenting cells that are able to capture skin antigens, prepare them and present them to T lymphocytes. This presentation leads to activation of the Th2 response, which results in the production of inflammatory cytokines such as IL-4, IL-5 and IL-13 (see for example Bieber, Ann Dermatol. 2010, 22(2): 125-137).

Once the individual has been sensitized cutaneously, subsequent contact with the allergen in question may induce eczema lesions. This response is also mediated by the Th2 response. In particular, Langerhans cells present the peptides to specific T lymphocytes that, when activated, produce Th2 cytokines (IL-4, IL-5). The resulting cytokines will recruit new cells, including eosinophils, which play an important role in the development and chronicity of eczema lesions.

In all periods of activity of the disease, bacterial or viral skin superinfections are the most common complications. The skin of atopic dermatitis patients is highly susceptible to secondary infections, which then tend to become more widespread. For example, the bacterium Staphylococcus aureus is a major cause of skin infections. It commonly colonizes the skin of atopic dermatitis patients, whereas it is only transiently present on healthy skin. The bacterium then secretes virulence factors that further reduce the barrier function, exacerbating the disease and contributing to its chronicity. In addition, S. aureus is usually found in atopic dermatitis patients in the form of homogeneous biofilms, a form resistant to host defenses and treatments.

U.S. Pat. No. 10,175,230 to Laboratoires Expanscience discloses a method for assessing the effectiveness of a C7 sugar or derivative thereof in the prevention and/or treatment of at least one deficiency of the skin barrier of a subject.

U.S. Published Application No. 20190242880 to Laboratoires Expanscience discloses methods for evaluating the in vitro efficacy of formulations in preventing the effects of dehydration on children's skin.

French Published Application No. 2792728 to L'Oreal discloses a method of evaluating the effects of a product on epidermal lipogenesis that includes applying the product to the surface of a skin equivalent, measuring the variation of a marker of epidermal lipids, then making a comparison with a similar measurement for a control sample.

United States Patent Application No. 20020182112 to Unilever Home & Personal Care USA discloses an in vivo method for measuring the binding of chemical compounds or mixtures of compounds to skin constituents.

U.S. Pat. Nos. 9,808,408 and 10,172,771 to The Procter & Gamble Company discloses a method of identifying a rinse off personal care composition that includes: (a) generating one or more control skin profiles for two or more subjects; (b) contacting at least a portion of skin of the subjects with a rinse-off test composition, rinsing the test composition off the portion of skin, extracting one or more skin samples from each of the subjects, and generating from the extracted samples one or more test profiles for the subjects; (c) comparing the one or more test profiles to the one or more control profiles and identifying the rinse-off test composition as effective for improving the stratum corneum barrier in a human subject who shows (i) a decrease in one or more inflammatory cytokines, (ii) an increase in one or more natural moisturizing factors, (iii) an increase in one or more lipids, and (iv) a decrease in total protein. Ring J. (2016) Pathophysiology of Atopic Dermatitis/Eczema. In: Atopic Dermatitis. Springer, Cham PMID:16098026, discloses the state of the art in research in atopic dermatitis, or atopic eczema.

Glatz et al., Emollient use alters skin barrier and microbes in infants at risk for developing atopic dermatitis, PLoS ONE, 13(2):e0192443 (2018), discloses that emollient use correlated with an increased richness and a trend toward higher bacterial diversity as compared to no emollient use in infants at risk for developing atopic dermatitis.

Capone et al., Effects of emollient use on the developing skin microbiome, presented at the American Academy of Dermatology Annual Meeting, 1-5 Mar. 2019, Washington DC, USA, discloses that microbial richness is significantly greater with infant wash and lotion than with wash alone. Capone et al. also discloses that both cleansing alone and cleansing and emollient regimens were well tolerated and that mild infant wash+lotion routine may best help improve microbial richness, which may contribute to overall skin barrier health by providing the right environment for healthy skin microbes to flourish.

Røpke, Mads Almose, et al. “Non-invasive assessment of soluble skin surface biomarkers in atopic dermatitis patients-Effect of treatment.” Skin Research and Technology (2021), discloses the use of a non-invasive patch technique to collect chemokines and cytokines on the surface of lesional and non-lesional atopic dermatitis skin before and after topical treatment.

U.S. Pat. No. 8,053,003 to Laboratoires Expanscience discloses a method of treating sensitive skin, irritated skin, reactive skin, atopic skin, pruritus, ichtyosis, acne, xerosis, atopic dermatitis, cutaneous desquamation, skin subjected to actinic radiation, or skin subjected to ultraviolet radiation, comprising administering an effective amount of a composition comprising furan lipids of plant oil and thereby increasing synthesis of skin lipids.

U.S. Pat. No. 10,226,499 to KAMEDIS LTD. and BIO-FD&C CO. LTD. discloses a method of treating atopic dermatitis that comprises administering a therapeutically effective amount of a composition comprising water extracts of Rheum palmatum, Cnidium Monnieri, Scutellaria baicalensis, Sanguisorbae officinalis, and Ailanthus altissima to upregulate expression of a human beta-defensin in a cell of the subject.

U.S. Published Application No. 20100016232 to Novozymes A/S discloses a method for treating an inflammatory disease such as atopic dermatitis that comprises administering a human beta-defensin, including human beta defensin 1.

U.S. Published Application No. 20110217249 to Dreher discloses a method for treating skin diseases and disorders associated with deregulation of the skin's antimicrobial peptide formation, processing, or both comprising administering an effective amount of one or more antimicrobial peptide sequestering compounds to a patient suffering therefrom, wherein the disease or disorder may be atopic dermatitis and wherein the antimicrobial peptide being sequestered by the compound may be a human defensin polypeptide.

U.S. Pat. No. 11,090,393 to Johnson & Johnson Consumer Inc. discloses methods of evaluating the potential impact of a surfactant system on infant skin that includes use of a computational model of adult skin penetration to visualize penetration of a marker by optimizing penetration parameters so that the model of adult skin penetration profiles match the experimental data; and transferring the optimized penetration parameters to a computational model of infant skin.

U.S. Published Application No. 20200360259 to Johnson & Johnson Consumer Inc. discloses a method of screening a skin treatment regimen, ingredient and/or composition for benefit to skin that includes measuring the level of one or more small molecule metabolites in an area of skin prior to application of the skin treatment regimen, ingredient and/or composition.

U.S. Pat. No. 11,229,595 to Johnson & Johnson Consumer Inc. discloses a method of evaluating an ability of a skin barrier system to protect infant skin from external irritants that includes use of a computational model of adult skin inflammation to visualize an effect of an external irritant by optimizing inflammation parameters so that the model of adult skin inflammation profiles match the experimental data; and transferring the optimized inflammation parameters to a computational model of infant skin.

Corresponding U.S. Patent Application No. 63/304,290 to Johnson & Johnson Consumer Inc. discloses factors that can be used to predict the propensity of an individual to develop atopic dermatitis. The references also discloses methods of using the factors to evaluate the potential of a skin treatment regimen, ingredient and/or composition to prevent atopic dermatitis.

Although daily use of moisturizers is included in many published guidelines for atopic dermatitis treatment (see, e.g., Hanifin et al., Guidelines of care for atopic dermatitis, J Am Acad Dermatol. 2004), there is no unanimous opinion in the scientific and medical community whether moisturizers should be used on newborns to prevent atopic dermatitis. See, e.g., FIG. 3, which sets out a brief summary of studies conducted on the use of emollients to prevent atopic dermatitis, which shows that:
two pilot studies show a 30-50% reduction of atopic dermatitis onset in babies at risk (Japan (Reference No. 8)¹ and US (Reference No. 16), respectively); ¹Citation of references appears at the end of this disclosure.
two large studies (BEEP* (Reference No. 2) and Prevent ADALL* (Reference No. 17)) did not show positive results (UK, Norway Multicentric) (see further discussion below);
several meta-analyses conclude no benefit of emollient use on prevention or delaying of atopic dermatitis (see, e.g., Reference No. 20);
one study showed around 50% reduction with different moisturizers (Reference No. 18).
Galderma announced that Cetaphil Lotion emollient therapy on newborns did not prevent or reduce atopic dermatitis in newborns at risk (Reference No. 13);
Pilot data in another large ongoing study (PEBBLES**) showed no significant effect at 6 and 12 months (Reference No. 12 (see also discussion below)).

As a result, today, many in the scientific community tend to believe that moisturizer use on newborns show no effect or can have a negative impact.

To date, there is no cure for atopic dermatitis. Treatments are primarily local, the aim of which is to improve symptoms and control disease progression (Eichenfield et al., J Am Acad Dermatol. 2014; 70(2): 338-351; Eichenfield et al., J Am Acad Dermatol. 2014; 71(1): 116-132). Many different emollients are available on the market. However, the precise mechanisms by which they exert their effects are insufficiently understood. There thus remains a need to further understand the mechanisms and to select effective, well-tolerated emollients to not only treat, but hopefully also prevent atopic dermatitis.

### SUMMARY OF THE INVENTION

The window of opportunity skin treatment regimen and composition will be described in more detail below.

It is believed that the window of opportunity regimen and composition exerts its effect at least in part by a combination of timing of treatment in the regimen and action of a single ingredient in the composition; and/or by a combination of timing of treatment in the regimen and action of a combination of two or more ingredients in the composition; and/or by a combination of timing of treatment in the regimen and action based on the amount of a single ingredient in the composition; and/or by a combination of timing of treatment in the regimen and action based on the relative amounts of two or more ingredients in the composition.

It is believed that the window of opportunity regimen and composition exerts its effect by one or more of the following:
a. Improving barrier function;
b. Having a probiotic effect on skin microbiome, thereby stimulating the growth of microorganisms having beneficial properties;
   i. It is believed that some of the ingredients may achieve this by acting as a buffer to achieve optimal skin pH.
c. Having an antioxidant effect;
d. Having an anti-inflammatory effect;
e. Stimulating ceramide production;
f. Stimulating fatty acid production.

The window of opportunity composition may comprise one or more of the following ingredients, preferably in the following amounts:
a. caprylic/capric triglyceride, preferably from about 0.0% to about 1.0%, preferably from about 0.005% to about 1.0%;
b. citric acid, preferably from about 0.0% to about 0.1%, preferably from about 0.005% to about 0.1%;
c. benzoic acid, preferably from about 0.0% to about 2.0%, preferably from about 0.1% to about 2.0%;
d. potassium phosphate, preferably from about 0.0% to about 2.0%, preferably from about 0.05% to about 2.0%;
e. dimethicone, preferably from about 0.0% to about 10.0%, preferably from about 1.0% to about 10.0%;
f. stearic acid; palmitic acid, preferably from about 0.0% to about 2.0%, preferably from about 0.1% to about 2.0%;
g. isocetyl alcohol, preferably from about 0.0% to about 10.0%, preferably from about 1.0% to about 10.0%;
h. Avena Sativa (Oat) Kernel Flour, preferably from about 0.0% to about 2.0%, preferably from about 0.1% to about 2.0%;
i. ceramide 3, preferably from about 0.0% to about 0.1%, preferably from about 0.005% to about 0.1%;
j. ethylhexylglycerin, preferably from about 0.0% to about 2.0%, preferably from about 0.1% to about 2.0%;
k. Avena Sativa (Oat) Kernel Oil, preferably from about 0.0% to about 1.0%, preferably from about 0.005% to about 1.0%;
l. Avena Sativa (Oat) Kernel Extract, which includes glycerin, potassium sorbate and water, preferably from about 0.0% to about 1.0%, preferably from about 0.005% to about 1.0%;
m. water, preferably from about 15.0% to about 40.0%, preferably from about 20.0% to about 40.0%;
n. glycerin, preferably from about 20.0% to about 50.0%, preferably from about 30.0% to about 50.0%;
o. sodium cetearyl sulfate, preferably from about 0.0% to about 2.0%, preferably from about 0.1% to about 2.0%;
p. dipotassium phosphate, preferably from about 0.0% to about 2.0%, preferably from about 0.1% to about 2.0%;
q. sodium hydroxide, preferably from about 0.0% to about 1.0%, preferably from about 0.005% to about 1.0%;
r. cetyl alcohol, preferably from about 0.0% to about 10.0%, preferably from about 1.0% to about 10.0%;
s. cetearyl alcohol, preferably from about 0.0% to about 10.0%, preferably from about 1.0% to about 10.0%;
t. benzyl alcohol, preferably from about 0.0% to about 2.0%, preferably from about 0.1% to about 2.0%;
u. p-anisic acid, preferably from about 0.0% to about 2.0%, preferably from about 0.1% to about 2.0%.

The ingredients alone or combined with other ingredients are believed to exert their effects at least in part as follows:
a. Improving barrier function;
   i. Caprylic/capric triglyceride creates a barrier on the skin's surface, which helps to reduce skin dryness by decreasing the loss of moisture.
   ii. Dimethicone acts as a skin protectant.
   iii. Stearic acid and palmitic acid are fatty acids that create an occlusive barrier that protects skin from unwanted microbes and pollutants while retaining moisture.
   iv. Hexyldecanol is a fatty alcohol that forms a film over the skin surface.
   v. Ceramide, together with saturated fatty acids, creates a water-impermeable, protective layer to prevent excessive water loss due to evaporation as well as a to provide a barrier against the entry of microorganisms.
   vi. Glycerin also known as glycerol is a humectant that can hydrate the outer layer of the skin and improve skin barrier function and skin mechanical properties.
b. Having a probiotic effect on skin microbiome, thereby stimulating the growth of microorganisms having beneficial properties. It is believed that the application of one or more of the ingredients in the composition may play a role in the balance of the skin microbiota.
   i. Oatmeal-based products work with skin to do a number of things, including creating a protective barrier, known as the skin microbiome, on the surface of the skin that guards against opportunistic pathogenic microorganisms.
      1. Avena Sativa (Oat) Kernel Flour
      2. Avena Sativa (Oat) Kernel Oil
      3. water; glycerin; Avena Sativa (Oat) Kernel Extract
   ii. Buffer skin pH to help skin achieve optimal pH levels that can affect the survival and growth rates of microbial species:
      1. citric acid
      2. benzoic acid
      3. potassium phosphate
      4. dipotassium phosphate
      5. sodium hydroxide
   iii. Glycerin also known as glycerol is a humectant that can have an effect on the microbiome.
   iv. Having an antioxidant effect;
      1. Caprylic/capric triglyceride can act as an antioxidant, which work to neutralize toxins exposed to in an environment.
      2. Tocopherol
   v. Having an anti-inflammatory effect;
      1. One or more ingredients in the composition may correct subclinical skin barrier dysfunction and early inflammation in predisposed infants before atopic dermatitis development by improving skin hydration and reducing skin permeability. This skin barrier enhancement prevents skin dryness and cracking, as well as inhibiting irritant and allergen penetration into the epidermis, which are potential initiators of skin inflammation.
         a. Oat ingredients have anti-inflammatory qualities that help to reduce itchiness.
         i. Avena Sativa (Oat) Kernel Flour
         ii. Avena Sativa (Oat) Kernel Oil
         iii. water; glycerin; Avena Sativa (Oat) Kernel Extract
   vi. Stimulating ceramide production;
      1. Avena Sativa (Oat) Kernel Flour
      2. Avena Sativa (Oat) Kernel Oil
      3. water; glycerin; Avena Sativa (Oat) Kernel Extract
      4. Glycerin also known as glycerol
   vii. Stimulating fatty acid production.
      1. Glycerin also known as glycerol

Ethylhexylglycerin is a glyceryl ether that is commonly used as part of a preservative system or its skin-conditioning properties in cosmetic preparations.

Sodium cetearyl sulfate is a mixture of stearyl and cetyl sulfate that functions as a surfactant in compositions.

Cetyl alcohol serves as a thickening agent and emulsifier, to help keep product ingredients from separating.

Cetearyl alcohol is a mixture of cetyl alcohol and stearyl alcohol, both fatty alcohols, and are used in personal care products, to create smoother, thicker compositions.

Benzyl alcohol acts as a preservative in skincare products due to its antibacterial and anti-fungal properties.

P-Anisic acid acts as a masking agent (meaning that it helps to mask smells in the product) and as a preservative.

Dimethicone may also have a defoaming effect, which could affect the aesthetics of the product to make it more desirable for use by a consumer.

### DESCRIPTION OF THE FIGURES

FIG. 1 is a graph showing point prevalence and cumulative incidence of atopic dermatitis (AD) at 6 and 12 months.

FIG. 2 is a graph showing prevalence of atopic dermatitis (AD) at 6 and 12 months for the total population; the intervention group; and the control group.

FIG. 3 is a table that sets out a brief summary of studies conducted on the use of emollients to prevent atopic dermatitis.

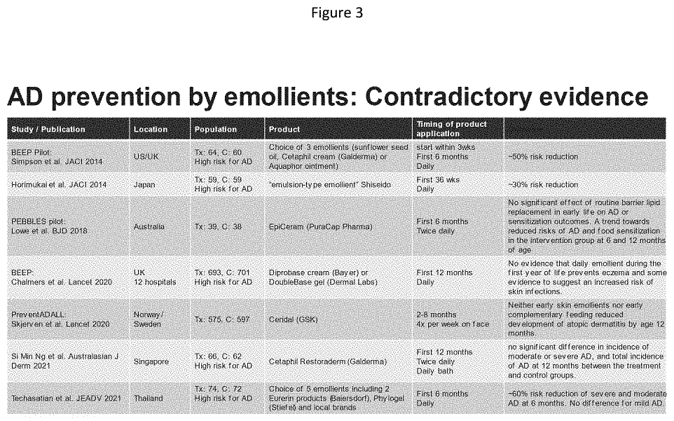

### DETAILED DESCRIPTION

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As used herein, the following terms shall have the meaning specified thereafter:

“Biomarker” as used herein refers to any biological molecule (gene, protein, lipid, metabolite) that, singularly or collectively, reflect the current or predict future state of a biological system. Thus, as used herein, various biomarkers are indicators of the quality of skin. The ability to prevent the onset of and/or treat skin conditions can also be assessed by measuring one or more biomarkers.

“Carrier” means a medium that is useful in preparing a composition that is generally compatible with the other ingredients in the composition, not deleterious to the recipient, and neither biologically nor otherwise undesirable.

“C-C motif Chemokine 2” (“CCL-2”) regulates cellular mechanics and thereby recruits monocytes, memory T cells, and dendritic cells to the sites of inflammation produced by either tissue injury or infection.

“C-C Motif Chemokine 22” and “C-C Motif Chemokine 17” (“CCL-22” and CCL-17) are Th2 chemokines that play important roles in the pathogenesis of atopic dermatitis.

“Composition” as used herein refers the ingredients or constituents that make up a mixture. Composition may include a single ingredient with an acceptable carrier.

“Consumer” as used herein refers to an individual who purchases and/or uses skin treatment regimens and compositions in accordance with the disclosure. In some instances, therefore, a consumer may be alternately referred to herein as a “user.”

“Cumulative incidence” is calculated as the number of new events or cases of disease divided by the total number of individuals in the population at risk for a specific time interval. Point prevalence differs from cumulative incidence in that point prevalence includes all cases, both new and preexisting, in the population at the specified time, whereas cumulative incidence is limited to new cases only. Incidence conveys information about the risk of contracting the disease, whereas prevalence indicates how widespread the disease is.

“Effective amount” as used herein means an amount of a regimen and composition sufficient to significantly induce a positive skin benefit, including independently or in combination with other benefits disclosed herein. This means that the content and/or concentration of active component in the regimen and composition is sufficient that when the regimen and composition is applied with normal frequency and in a normal amount, the regimen and composition can result in the prevention of the onset of or treatment of one or more undesired skin conditions. For instance, the amount can be an amount sufficient to inhibit or enhance some biochemical function occurring within the skin.

“Emollient” as used herein refers to chemical agents specially designed to make the external layers of the skin (epidermis) softer and more pliable.

“Epidermis” as used herein refers to the outer layer of skin, and is divided into five strata, which include the: stratum corneum, stratum lucidum, stratum granulosum, stratum spinosum, and stratum basale. The stratum corneum contains many layers of dead, anucleated keratinocytes that are essentially filled with keratin. The outermost layers of the stratum corneum are constantly shed, even in healthy skin. The stratum granulosum contains two to four layers of cells that are held together by desmosomes that contain keratohyaline granules. The stratum spinosum contains eight to ten layers of smaller cells that are also held together by desmosomes. The stratum basale contains a single layer of columnar cells that actively divide by mitosis and provide the cells that are destined to migrate through the upper epidermal layers to the stratum corneum. The predominant cell type of the epidermis is the keratinocyte. These cells are formed in the basal layer and exist through the epidermal strata to the granular layer at which they transform into the cells know as corneocytes or squames that form the stratum corneum. During this transformation process, the nucleus is digested, the cytoplasm disappears, the lipids are released into the intercellular space, keratin intermediate filaments aggregate to form microfibrils, and the cell membrane is replaced by a cell envelope made of cross-linked protein with lipids covalently attached to its surface. Keratins are the major structural proteins of the stratum corneum. Corneocytes regularly slough off (a process known as desquamation) to complete an overall process that takes about a month in healthy human skin. In stratum corneum that is desquamating at its normal rate, corneocytes persist in the stratum corneum for approximately 2 weeks before being shed into the environment.

“Epithelial tissue” as used herein refers to all or any portion of the epithelia, in particular the epidermis, and includes one or more portions of epithelia that may be obtained from a subject by a harvesting technique known in the art, including those described herein. By way of example and without being limiting, epithelial tissue refers to cellular fragments and debris, proteins, isolated cells from the epithelia including harvested and cultured cells.

“Filaggrin” (filament aggregating protein) as used herein refers to a filament-associated protein that binds to keratin fibers in epithelial cells. Filaggrin is essential for the regulation of epidermal homeostasis. Within the stratum corneum, filaggrin monomers can become incorporated into the lipid envelope, which is responsible for the skin barrier function. Alternatively, these proteins can interact with keratin intermediate filaments. Filaggrin undergoes further processing in the upper stratum corneum to release free amino acids that assist in water retention. It is believed that filaggrin is an important player in the pathogenesis of atopic dermatitis and allergic disease.

Human-beta-defensin 1 (hBD1) is an antimicrobial peptide constitutively expressed by epithelial cells at mucosal surfaces and in the epidermis.

“Infant” as used herein refers to a human whose age ranges from birth to approximately twelve months of life.

“Inflammatory cytokine” is a type of signaling molecule that is secreted from immune cells and certain other cell types that promotes inflammation. Inflammatory cytokines are predominantly produced by T helper cells (Ths) and macrophages and involved in the upregulation of inflammatory reactions.

“Interleukin-1 family” (“IL-1 family”) is a group of 11 cytokines that plays a central role in the regulation of immune and inflammatory responses to infections or sterile insults.

“Interleukin-1A” (“IL-1A”), which is produced by epithelial cells, plays an important role in immune responses.

“Interleukin-1B” (“IL-1B”) is a cytokine of the IL-1 family, an important mediator of the inflammatory response, produced in skin by keratinocytes and immune cells.

“IL-1 receptor antagonist” (“IL-1RA”) is a natural antagonist of family members of IL-1.

“Interleukin-8” (“IL-8”) is a potent chemotactic and proinflammatory cytokine, produced in the skin by a variety of cells in response to inflammatory stimuli.

“Interleukin-18” (“IL-18”) also known as interferon-gamma inducing factor, is a pro-inflammatory cytokine produced in keratinocytes.

“Interleukin-36 gamma” (“IL-36gamma”) is cytokine in the IL-1 family with pro-inflammatory effects.

“Metabolite” as used herein refers to the intermediate end product of metabolism. The term metabolite is usually restricted to small molecules. Metabolites have various functions, including fuel, structure, signaling, stimulatory and inhibitory effects on enzymes, catalytic activity of their own (usually as a cofactor to an enzyme), defense, and interactions with other organisms (e.g., pigments, odorants, and pheromones). A primary metabolite is directly involved in normal “growth”, development, and reproduction. A secondary metabolite is not directly involved in those processes, but usually has an important ecological function.

“Mixture” as used herein is the result of two or more substances that are not chemically combined.

“Moisturizers” are chemical agents that add moisture to the skin.

“Package” includes any suitable container for personal care regimens and compositions.

“Personal care composition” as used herein, refers to compositions intended for topical application to the skin. The compositions used in accordance with the present disclosure include topically applied compositions. The personal care composition used in accordance with the present disclosure is typically dispensable from a package. Thus, in some embodiments, the dispensing may be by extruding. In some embodiments the package may be a single chamber package, or a multi chamber package, or a set of discrete packages. The personal care compositions used in accordance with the present disclosure can be in the form of a balm, a cream, a lotion, an emulsion, a serum, an ointment and a paste, intended for topical application to skin.

“Point prevalence” means the prevalence measured at a particular point in time. It is the proportion of persons with a particular disease or attribute on a particular date.

“P-value” is a measure of the probability that an observed difference could have occurred just by random chance. The lower the p-value, the greater the statistical significance of the observed difference. P-value can be used as an alternative to or in addition to pre-selected confidence levels for hypothesis testing. The standard level of significance used to justify a claim of a statistically significant effect is 0.05. The term statistically significant has become synonymous with p 0.05. Bross I D J (1971), “Critical Levels, Statistical Language and Scientific Inference,” in Godambe V P and Sprott (eds) Foundations of Statistical Inference. Toronto: Holt, Rinehart & Winston of Canada, Ltd.

“16S rRNA gene sequencing” is a methodology used for identification, classification and quantitation of microbes within complex biological mixtures.

“Skin” is divided into three main structural layers, the outer epidermis, the inner dermis, and the subcutaneous tissue.

“Stratum corneum” as used herein, refers to the outermost layer of the epithelia, or the epidermis, and is the skin structure that provides a chemical and physical barrier between the body of an animal and the environment. The stratum corneum is a densely packed structure comprising an intracellular fibrous matrix that is hydrophilic and able to trap and retain water. The intercellular space is filled with lipids formed and secreted by keratinocytes and which provide a diffusion pathway to channel substances with low solubility in water.

“Subject” as used herein refers to a human for whom a regimen and composition is tested or on whom a regimen and composition is used in accordance with the methods described herein.

“Substantially free of” as used herein, unless otherwise specified, means that the personal care regimen and composition comprises less than about 2%, less than about 1%, less than about 0.5%, or even less than about 0.1% of the stated ingredient. The term “free of”, as used herein, means that the personal care regimen and composition comprises 0% of the stated ingredient. However, these ingredients may incidentally form as a by-product or a reaction product of the other components of the personal care regimen and composition.

“Topical application”, “topically”, and “topical”, as used herein, mean to apply the regimen and composition used in accordance with the present disclosure onto the surface of the skin.

“Treating” or “treatment” or “treat” as used herein includes regulating and/or immediately improving skin appearance and/or feel. Treating may include secondary prevention, which protects against disease exacerbation.

“Vascular endothelial growth factor A” or “VEGF-A” is a mediator of vascular hyperpermeability, angiogenesis, and inflammation, processes intimately involved in tissue repair.

“Young child/children” as used herein refers to a human/humans whose age ranges from approximately twelve months of life to approximately 3 years, or approximately 5 years, or approximately 7 years of life.

### EXAMPLE

STOP AD (Short-term Topical Application for Prevention of Atopic Dermatitis), a randomized, open-label, controlled study designed to investigate the effect of short-term neonatal skin barrier protection using the skin treatment regimen and composition below on the prevention of atopic dermatitis in high-risk infants, was conducted. (See https://clinicaltrials.gov/ct2/show/NCT03871998; ucc.ie/en/paediatrics/programmes/postgraduateprorammes/phd-students/; and https://www.infantcentre.ie/research/research-studies/stop-ad/) and Ni Chaoimh, et al., Parental compliance with an infant moisturization protocol in the first 2 months of life; Lad et al., Can more be done to implement translational weaning advise for new mothers; and Lad et al., Neonatal natural moisturizing factor concentrations in a high-risk cohort with parental history of atopy compared to a reference cohort, each presented at European Academy of Allergy and Clinical Immunology (EAACI) 2020). High-risk infants were identified using parental history of atopic disease and were randomized to either the skin treatment regimen and composition or standard routine care, each set forth below, from soon after recruitment in the postnatal ward (approximately 4 day) until eight weeks of age. Both the intervention and standard of care groups were advised to use a standardized bathing routine for the first 2 months.
Skin treatment regimen and composition: twice daily whole-body application of AVEENO® Dermexa Fast & Long-Lasting Balm, which on pack contains the following ingredients: glycerin, aqua, cetearyl alcohol, isocetyl alcohol, dimethicone, cetyl alcohol, Avena sativa kernel flour, Avena sativa kernel extract, Avena sativa kernel oil, caprylic/capric triglyceride, ceramide 3, ethylhexylglycerin, p-anisic acid, sodium cetearyl sulfate, palmitic acid, stearic acid, sodium sulfate, sodium chloride, citric acid, dipotassium phosphate, potassium phosphate, sodium hydroxide, tocopherol, benzyl alcohol, benzoic acid, and potassium sorbate.
Control: Standard routine care, which involves no use of moisturizer for the first two months.

Both groups received: AVEENO® Baby Daily Care Gentle Wash, which on pack contains the following ingredients: aqua, glycerin, cocamidopropyl betaine, sodium lauroamphoacetate, coco-glucoside, sodium chloride, hydroxypropyl starch phosphate, Avena sativa kernel flour, Aloe barbadensis leaf juice, Olea europaea leaf extract, Chamomilla recutita extract, Helianthus annuus seed oil, sarcosine, magnesium aspartate, potassium aspartate, polyquaternium-7, polysorbate 20, sodium cocoyl amino acids, acrylates/C10-30 alkyl acrylate crosspolymer, propylene glycol, citric acid, sodium hydroxide, tocopherol, tocopheryl acetate, sodium benzoate, potassium sorbate, sodium sulfite, parfum.

Infants with at least one parent with a positive history of atopic disease (AD, allergic rhinitis or asthma) were eligible for recruitment. Additional inclusion or exclusion criteria are set forth below.
Inclusion Criteria:
   Healthy full-term infants, gestational age >36+6 weeks.
   At least one parent with self-reported atopic dermatitis, food allergy, allergic rhinitis or asthma.
   Not require admission to the neonatal unit.
Exclusion Criteria:
   No parental history of atopic disease.
   Require admission to the neonatal unit for issues other than the establishment of normal feeding.
   Having been administered oral or parenteral antibiotics.
   Receiving phototherapy for hyperbilirubinaemia.
   Sibling, including twin, already recruited.
   Other serious health issues (e.g., abdominal wall defects, congenital heart disease etc.) or a severe widespread skin condition (e.g., collodion).
   Any condition that would make the use of skin barrier protectant inadvisable or not possible (e.g., ankle talipes or developmental dysplasia of the hip, requiring a Pavlik's harness or casts).
   Participation in any other clinical trial of an investigational medicinal product.

Within approximately 4 days of birth, infants were randomized to either treatment with skin treatment regimen and composition or to standard routine skincare with no moisturizer until 2 months of age. 260 infants participated in the study, including 120 in the intervention group and 140 in the control group.

The study had six visits during the first year of life, within 4 days of birth and at 2, 4 and 8 weeks and 6 and 12 months, involving repeat measurements of weight, trans-epidermal water loss (TEWL) and Raman-derived natural moisturizing factor (NMF) to assess skin barrier function and structure in addition to monitoring of skin health and feeding. A questionnaire on infant health, bathing, feeding and skincare was filled out and skin swabs were taken for microbiome and immune biomarker analysis.

The study's primary outcome was the effect of the intervention on the incidence of atopic dermatitis at 12 months. Secondary outcomes included the effect of the intervention on the incidence of atopic dermatitis at 6 months and the evolution of TEWL and NMF values from 0-12 months.

Skin swabs were taken at baseline and again at 8 weeks and at 12 months. A healthcare worker blind to treatment allocation assessed the presence (yes/no), extent and severity of atopic dermatitis at 6 and 12 months. A DNA sample was taken to test for filaggrin loss-of-function mutations, which are linked to atopic dermatitis risk.

I-SEAL (Insights towards understanding Skin Function in Early Life) involved the collection of skin microbiome and immunity biomarkers within the larger intervention trial STOP AD in order:
to examine the temporal transition of the skin microbiome between birth and 12 months and its influence on the development of atopic dermatitis.
to investigate the impact of the use of the skin regimen and composition in the first two months of life on infant skin microbiota.
to investigate the dynamics of immunity biomarkers collected from the surface of infant skin in the first 12 months, and to examine associations with atopic dermatitis.

Table 1 presents the parent-reported moisturization frequency in the control and intervention groups. These data indicate good compliance with study allocation and also support the feasibility of adopting daily moisturization into the skin care routine in the first 2 months of life.

TABLE 1

| | Control (n = 141) | | | Intervention (n = 121) | | |
|---|---|---|---|---|---|---|
| | 2 weeks | 4 weeks | 8 weeks | 2 weeks | 4 weeks | 8 weeks |
| | (n = 137) | (n = 137) | (n = 141) | (n = 118) | (n = 120) | (n = 119) |
| Never | 47 (34.3) | 39 (28.5) | 34 (24.1) | 0 | 0 | 0 |
| Occasionally | 25 (18.2) | 25 (18.2) | 25 (17.7) | 1 (0.8) | 0 | 0 |
| Once/week | 7 (5.1) | 9 (6.6) | 10 (13.5) | 1 (0.8) | 0 | 2 (1.7) |
| 2-3/week | 32 (23.4) | 40 (29.2) | 44 (31.2) | 3 (2.5) | 3 (2.5) | 5 (4.2) |
| 4-6/week | 16 (11.7) | 17 (12.4) | 11 (7.8) | 8 (6.8) | 6 (5.0) | 9 (7.6) |
| Daily | 10 (7.3) | 7 (5.1) | 8 (5.7) | 105 (89.0) | 111 (91.7) | 103 (86.6) |
| Twice/day | 2 (1.5) | 0 | 0 | 75 (63.6) | 83 (69.2) | 87 (73.1) |

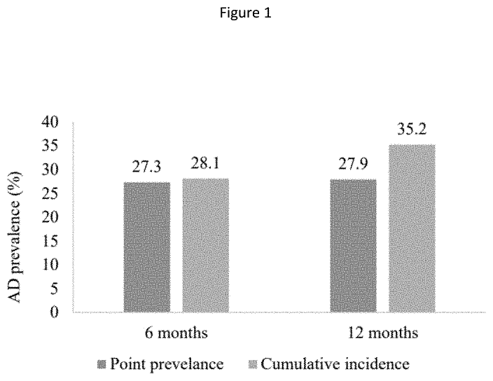

103 participants were diagnosed with atopic dermatitis. FIG. 1 presents the point prevalence and cumulative incidence of atopic dermatitis at 6 and 12 months. The prevalence of atopic dermatitis was 27.3% and 27.9% at 6 and 12 months, respectively, and 17.0% participants met the UK Working Party Diagnostic Criteria, Williams et al., Br J Dermatol. 1994 September; 131(3):406-16. doi: 10.1111/j.1365-2133.1994.tb08532.x., at both 6 and 12 months.

### Interim Analysis at 6 Months

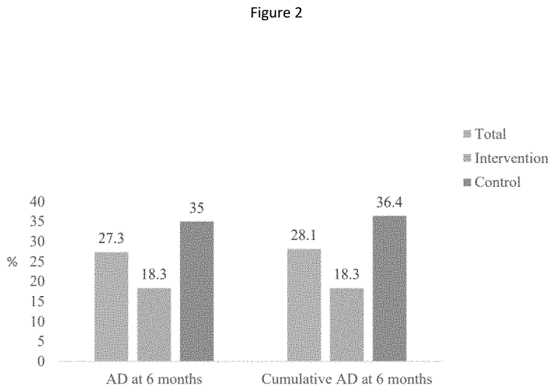

Of the 262 participants, 260 (120 intervention and 140 control) were included in the interim analysis at 6 months. Two participants, one from the intervention group and one from the control group, were excluded from the analysis due to missing data on atopic dermatitis outcome at 6 months. Baseline characteristics were distributed evenly across study groups. In the total cohort, the point prevalence and cumulative incidence of atopic dermatitis at 6 months was 27.3% and 28.1%, respectively. The cumulative incidence of atopic dermatitis was 18.3% in the intervention group and 36.4% in the control group [relative risk (RR): 0.503, 95% CI: 0.325, 0.779] corresponding to a 50% decreased risk of atopic dermatitis at 6 months in the intervention group, see FIG. 2.

### Filaggrin Analysis

DNA samples for the analysis of filaggrin gene status were collected using buccal swabs for filaggrin genotyping.

Skin swabs for microbiome and immune biomarker analysis were taken at baseline, at the end of the intervention period (8 weeks) and at 12 months from study participants. Swabs were taken from two skin sites: the cheek and the antecubital fossa (elbow pit). Skin swabs from a subgroup of approximately 30 infants from each study group (control and intervention) were sent to CosmosID, Rockville, MD, USA for analysis at the end of the 12-month assessments.

Swabs taken for microbiome analysis at the end of sample collection at 12 months were analyzed as a single batch for common 16s rRNA gene sequencing.

### Immunity Biomarkers

In addition to the microbiome swabs, samples from the same subgroup identified for microbiome analysis were sent for analysis at the end of sample collection. Samples for the quantification of immunity biomarkers were taken from the skin areas of interest at baseline, 8 weeks and 12 months using specialized swabs soaked in buffer (FibroTx, Tallinn, Estonia). Samples were stored on dry ice until further processing and at −80° C. until shipment to FibroTx for marker analysis using a spot enzyme-linked immunosorbent assay. The samples were analyzed for the presence of IL-1A, IL-1RA, VEGF-A, IL-8, IL-1B, IL-18, CCL-22, CCL-17, CCL-2 and hBD-1.

An interim analysis of the 6-month data showed a 50% decreased risk of atopic dermatitis in those who moisturized from birth to 8 weeks compared to the control group. This contrasts with recent findings in two large, randomised control trials, BEEP and PreventADALL, where no evidence of a protective effect of emollient use in the first year was found.

Chambers et al., Daily emollient during infancy for prevention of eczema: the BEEP randomized controlled trial, Lancet 2020; 395: 962-72. Published Online February 19, 2020, https://doi.org/10.1016/S0140-6736(19)32984-8, discloses that the investigators found no evidence that daily emollient during the first year of life prevents eczema in high-risk children and that there was some evidence to suggest that daily emollient use caused an increased risk of skin infections. In accordance with the study, term newborns with a family history of atopic disease were randomly assigned (1:1) to application of emollient daily (either Diprobase cream or DoubleBase gel) for the first year plus standard skin-care advice (emollient group) or standard skin-care advice only (control group). Diprobase cream contains white soft paraffin, cetostearyl alcohol, liquid paraffin, macrogol cetostearyl ether, chlorocresol, sodium dihydrogen phosphate, sodium hydroxide, phosphoric acid, and purified water. DoubleBase gel contains liquid paraffin, isopropyl myristate, glycerol, carbomer, sorbitan laurate, trolamine, phenoxyethanol and purified water.

Skjerven et al., Skin emollient and early complementary feeding to prevent infant atopic dermatitis (PreventADALL): a factorial, multicentre, cluster-randomised trial, Lancet. 2020 Mar. 21; 395(10228):951-961. doi: 10.1016/S0140-6736(19)32983-6. Epub 2020 Feb. 19, discloses that the investigators found that neither early skin emollients nor early complementary feeding reduced development of atopic dermatitis by age 12 months. The skin intervention consisted of baths for 5-10 min with added emulsified oil (0.5 dL of bath oil per 8 L of water) and cream applied to the entire face after the bath (Ceridal; GlaxoSmithKline Consumer Healthcare, Philadelphia, PA, USA) on at least 4 days per week, from week 2 to age 8 months. Parents were instructed at the maternity ward on safe baby handling during bathing, including written instructions with illustrations. Ceridal bath oil contains Paraffinum liquidum, vitis vinifera, laureth-4, sorbitan sesquioleate, dicocodimonium chloride, BHT. Ceridal cream contains aqua, olea europaea, glycerin, pentylene glycol, palm glycerides, olus, hydrogenated lecithin, squalane, betaine, palmitamide MEA, acetamide MEA, sarcosine, hydroxyethylcellulose, sodium carbomer, carbomer, and xanthan gum.

In BEEP, the majority of infants (89%) were randomised by 21 days (median 11 days) and in PreventADALL, the skin intervention involved regular bath oil emollient and facial cream from 2 weeks of age.

It will be understood that, while various aspects of the present disclosure have been illustrated and described by way of example, the invention claimed herein is not limited thereto, but may be otherwise variously embodied according to the scope of the claims presented in this and/or any derivative patent application.

### SELECTED REFERENCES

1. Capone et al., Effects of emollient use on the developing skin microbiome, presented at the American Academy of Dermatology Annual Meeting, 1-5 Mar. 2019, Washington DC, USA.
2. Chalmers J R, Haines R H, Bradshaw L E, Montgomery A A, Thomas K S, Brown S J, Ridd M J, Lawton S, Simpson E L, Cork M J, et al. Daily emollient during infancy for prevention of eczema: the BEEP randomised controlled trial. Lancet 2020; 395 (10228):962-972.
3. Chambers et al., Daily emollient during infancy for prevention of eczema: the BEEP randomized controlled trial, Lancet 2020; 395: 962-72. Published Online Feb. 19, 2020, https://doi.org/10.1016/S0140-6736(19)32984-8.
4. Eichenfield et al., J Am Acad Dermatol. 2014; 70(2): 338-351.
5. Eichenfield et al., J Am Acad Dermatol. 2014; 71(1): 116-132.
6. Glatz et al., Emollient use alters skin barrier and microbes in infants at risk for developing atopic dermatitis, PLoS ONE, 13(2):e0192443 (2018).
7. Hanifin et al., Guidelines of care for atopic dermatitis, J Am Acad Dermatol. 2004.
8. Horimukai K, Morita K, Narita M, Kondo M, Kitazawa H, Nozaki M, Shigematsu Y, Yoshida K, Niizeki H, Motomura K et al. Application of moisturizer to neonates prevents the development of atopic dermatitis. J Allergy Clin Immunol 2014; 134 (4); 824-30.e6.² ²The investigators concluded that daily application of moisturizer during the first 32 weeks of life reduces the risk of AD/eczema in infants. The product used, 2e [Douhet] emulsion; Shiseido, Tokyo, Japan, contains the following ingredients: water, butylene glycol, squalene, xylitol, hydrogenated polydecene, pentaerythrityl, tetraethylhexanoate, jojoba seed oil, isostearic acid, PEG-60 glyceryl stearate, dimethicone, PEG-5 glyceryl stearate, behenyl alcohol, batyl alcohol, carbomer, potassium hydroxide, sodium metaphosphate, tocopherol, and phenoxyethanol.
9. Kelleher M, Dunn-Galvin A, Hourihane JO'B, Murray D M, Campbell L E, McLean W H I, Irvine A D. Skin barrier dysfunction measured by transepidermal water loss (TEWL) at 2 days and 2 months predates and predicts atopic dermatitis at 1 year. J Allergy Clin Immunol 2015; 135 (4) 930-935.
10. Kelleher M M, Dunn-Galvin A, Gray C, Murray D M, Kiely M, Kenny L, McLean W H I, Irvine A D, Hourihane JO'BH. Skin barrier impairment at birth predicts food allergy at 2 years of age. J Allergy Clin Immunol 2016; 137(4):1111-6.e8.
11. Kelleher et al., Skin care interventions in infants for preventing eczema and food allergy (review), Cochrane database of systemic reviews, John Wiley & Sons, Ltd. 2021.
12. Lowe A J, et al. A randomized trial of a barrier lipid replacement strategy for the prevention of atopic dermatitis and allergic sensitization: the PEBBLES pilot study. BMC Dermatol. 2018;178(1):e19-e21.³ ³The investigators found that twice-daily prophylactic use of a ceramide-containing emollient, from the neonatal period (about three weeks) to 6 months of age, showed no significant effect of routine barrier lipid replacement in early life on atopic dermatitis or sensitization outcomes. The authors noted however, that there was a trend towards reduced risks of atopic dermatitis and food sensitization in the intervention group at 6 and 12 months of age. The emollient, EpiCeram™; PuraCap Pharmaceutical LLC, South Plainfield, NJ, U.S.A., contains physiological ratios of ceramides, cholesterol and free fatty acids, and has a slightly acidic pH (5.0).
13. Si Min N g et al., Moisturisers from birth in at-risk infants of atopic dermatitis—a pragmatic randomised controlled trial, Australasian Journal of Dermatology (2021) 62, e539—e545, doi: 10.1111/ajd.13703.⁴ ⁴The investigators found that early use of moisturisers in ‘at risk’ infants do not reduce the incidence of moderate-to-severe AD and overall incidence of AD in infancy. Participants in the treatment group were required to apply Cetaphil Restoraderm (PRO AD Derma) Skin Restoring Moisturizer (Galderma S A, LaTour-de-Peilz, Switzerland) twice daily, and bathe with Cetaphil Restoraderm (PRO AD Derma) Skin Restoring Wash (Galderma S A, La Tour-de-Peilz, Switzerland). The Cetaphil Restoraderm (PRO AD Derma) Skin Restoring Moisturizer is formulated with both ceramides and 2 filaggrin breakdown products: arginine and sodium pyrrolidone carboxylic acid.
14. O'Regan G M, Kemperman P M, Sandi lands A, Chen H, Campbell L E, Kroboth K, Watson R, Rowland M, Puppels G J, McLean W H, Caspers P J, Irvine A D. Raman profiles of the stratum corneum define 3 filaggrin genotype-determined atopic dermatitis endophenotypes. J Allergy Clin Immunol 2010; 126 (3); 574-80.el.
15. Perkin M R, Logan K, Marrs T, Radulovic S, Craven J, Boyle R J, Chalmers J R, Williams H C, Versteeg S A, van Ree R, Lack G, Flohr C. Association of frequent moisturizer use in early infancy with the development of food allergy, Journal of Allergy and Clinical Immunology, Volume 147, Issue 3, March 2021, 967-976.e1.
16. Simpson E L, Chalmers J R, Hanifin J M, Thomas K S, Cork, M J, McLean W H, Brown S J, Chen Z, Chen Y, Williams H C. Emollient enhancement of the skin barrier from birth offers effect atopic dermatitis prevention. J Allergy Clin Immunol 2014; 134 (4); 818-23.⁵ ⁵The investigators concluded that emollient therapy from birth represents a feasible, safe, and effective approach for atopic dermatitis prevention. Parents in the intervention group were offered a choice of 3 emollients of different viscosities (an oil, a cream/ gel, or an ointment) that had been selected based on previous data regarding their safety, tolerability, or barrier-protective qualities. In the United Kingdom emollient choices were sunflower seed oil (William Hodgson and Co, Congleton, United Kingdom), Doublebase Gel (Dermal Laboratories, Hitchin, United Kingdom), and liquid paraffin 50% in white soft paraffin. In the United States parents were offered the same sunflower seed oil as used in the United Kingdom, Cetaphil Cream (Galderma Laboratories, Fort Worth, Tex), or Aquaphor Healing Ointment (Beiersdorf, Chester, Ohio). Parents were asked to apply the emollient to the baby's entire body surface, with the exception of the scalp, starting as soon as possible after birth (within a maximum of 3 weeks) and continuing until the infant was 6 months of age.
17. Skjerven H O, Rehbinder E M, Vettukattil R, Granum V, Haugen G, Hedlin G, Landrø L, Marsland B J, Rudi K, Sjøborg K D et al. Skin emollient and early complementary feeding to prevent infant atopic dermatitis (PreventADALL): a factorial, multicentre, cluster-randomised trial. Lancet 2020; 385 (10228):951-961.
18. Techasatian et al., Effects of an emollient application on newborn skin from birth for prevention of atopic dermatitis: A randomized controlled study in Thai neonates, JEADV, Volume36, Issue1, January 2022; pages 76-83; first published: 21 September 2021 https://doi.org/10.1111/idv.17675⁶ ⁶The investigators concluded that the study found that applying emollients in high-risk neonates within 3 weeks of life had preventive effects against developing AD at 6 months of age in neonates living in a tropical climate (Thailand); that the optimal frequency of applying emollients for the prevention of AD in this population was “as needed,” depending on environmental factors and level of skin dryness, rather than daily routine application. The majority of the participants had low (1-3 days/week; 40 [54.05%]) to moderate (4-6 days/week; 34[45.95%]) adherence to moisturizer application, respectively. None of the participants applied moisturizers daily. Parents in the intervention group were offered a choice of five emollients as follows: (A) Ezerra® lotion (HOE Pharmaceuticals Sdn. Bhd., Selangor, Malaysia), (B) Eucerin® Omega Plus Extra Soothing (Beiersdorf Co., Ltd., Bangkok, Thailand), (C) Eucerin® Omega Soothing lotion (Beiersdorf Co., Ltd., Bangkok, Thailand), (D) Physiogel® A.I. restoring lipid balm (Stiefel Co., Ltd., Bangkok, Thailand) and (E) LyL® Hydrating moisturizer (Cosmaprof Co., Ltd., Bangkok, Thailand). Parents were asked to apply the emollient at least once daily to the baby's entire body surface (excluding the scalp), starting as soon as possible after birth (within a maximum of 3 weeks) and continuing until the infant was 6 months of age.(A) Ingredients on pack include water, dicaprylyl carbonate, glycerin, cetearyl alcohol, Persea gratissima (avocado) oil, trehalose, saccharide isomerate, tapioca starch, dimethicone, pentaerythrityl distearate, Rosa canina fruit oil, cetearyl glucoside, spent grain wax, Butyrospermum parkii (shea butter) extract, Argania spinosa kernel oil, polymethylsilsesquioxane, phenoxyethanol, ethylhexylglycerin, tocopheryl acetate, xanthan gum, sodium citrate, disodium EDTA.(B) Ingredients on pack include Aqua, Glycerin, Caprylic/Capric Triglyceride, Dimethicone, Pentaerythrityl Tetraisostearate, Triisostearin, Vitis Vinifera Seed Oil, Oenothera Biennis Oil, Cetyl Alcohol, Glyceryl Stearate, PEG-40 Stearate, Glycyrrhiza Inflata Root Extract, Ceramide NP, Decylene Glycol, Menthoxypropanediol, Citric Acid, Sodium Citrate, Tocopherol, Ascorbyl Palmitate, Trisodium EDTA, BHT, 1,2-Hexanediol, Phenoxyethanol.(C) The website indicates that this product is highly concentrated in fatty acids Omega-6 (Evening primrose oil and Grape Seed Oil) and Licochalcone (extract of licorice root).(D) Ingredients on pack include Aqua, caprylic/caprice triglyceride, glycerin, pentylene glycol, olea europaea fruit oil, butyrospermum parkii butter, cocos nucifera oil, hydrogenated lecithin, palmitamide MEA, dehydroxanthan gum, acrylates/C10-30 alkyl acrylate crosspolymer, sodium carborner, ceramide NP.(E) Currently unable to locate ingredient list.
19. Williams et al., UK Working Party Diagnostic Criteria, Br J Dermatol. 1994 September; 131(3):406-16. doi: 10.1111/j.1365-2133.1994.tb08532.x.
20. Kelleher et al., Skin care interventions in infants for preventing eczema and food allergy (review), Cochrane database of systemic reviews, John Wiley & Sons, Ltd. 2021.⁷ ⁷Kelleher et al. reviewed the literature and interviewed investigators to assess the effects of skin care interventions, such as emollients, for primary prevention of eczema and food allergy in infants. The authors concluded that skin care interventions such as emollients during the first year of life in healthy infants are probably not effective for preventing eczema, and probably increase risk of skin infection; and that further work was needed to understand whether different approaches to infant skin care might promote or prevent eczema and to evaluate effects on food allergy based on robust outcome assessments.
21. Nowicki et al. News about Emollients with Rhealba Oat Plantlets Extract: Prevention and Treatment of Atopic Dermatitis. J Clin Pediatr Neonatal Care 2019; 6(2):021.⁸ ⁸The reference discloses that a study was conducted to assess the tolerance of emollients containing Rhealba Oat plantlets extract in three galenic forms (balm, cream and lotion) in a group of neonates. The study involved 53 neonates with high-risk of AD, and skin products were applied once daily for three weeks. No adverse reaction to the product was observed. (Pierre Fabre, clinical study RV34241201650). The reference also discloses that the clinical study included 3 visits and the maximum duration of the study for each subject was 24 days. During Visit 1, on Day 1, newborns under 96 hours of life were included in the study, the 2^{nd} visit was set up 8±2 days after and finally the 3^{rd} visit took place 22±2 days following the beginning of the study.

1. A window of opportunity regimen and composition for preventing the onset of or treating atopic dermatitis in a subject, comprising:
administering a first composition to the subject at least once a day;
wherein the first composition is administered to the subject starting from within about four days after birth of the subject and continues to at least about two months after birth of the subject.

2. The window of opportunity regimen and composition of claim 1, wherein the first composition is administered at least twice a day to the subject.

3. The window of opportunity regimen and composition of claim 1, wherein the first composition is administered to the whole body of the subject.

4. The window of opportunity regimen and composition of claim 1, further comprising:
administering a second composition to the subject in accordance with standard routine care;
wherein the second composition is administered to the subject starting from within about four days after birth of the subject and continues to at least about two months after birth of the subject.

5. The window of opportunity regimen and composition of claim 4, wherein standard routine care is administration two to three times a week.

6. The window of opportunity regimen and composition of claim 4, wherein standard routine care is administration daily.

7. The window of opportunity regimen and composition of claim 1, wherein the first composition comprises one or more ingredients selected from the group consisting of glycerin, aqua, cetearyl alcohol, isocetyl alcohol, dimethicone, cetyl alcohol, Avena sativa kernel flour, Avena sativa kernel extract, Avena sativa kernel oil, caprylic/capric triglyceride, ceramide 3, ethylhexylglycerin, p-anisic acid, sodium cetearyl sulfate, palmitic acid, stearic acid, sodium sulfate, sodium chloride, citric acid, dipotassium phosphate, potassium phosphate, sodium hydroxide, tocopherol, benzyl alcohol, benzoic acid and potassium sorbate and a carrier.

8. The window of opportunity regimen and composition of claim 1, wherein the first composition comprises two or more ingredients selected from the group consisting of glycerin, aqua, cetearyl alcohol, isocetyl alcohol, dimethicone, cetyl alcohol, Avena sativa kernel flour, Avena sativa kernel extract, Avena sativa kernel oil, caprylic/capric triglyceride, ceramide 3, ethylhexylglycerin, p-anisic acid, sodium cetearyl sulfate, palmitic acid, stearic acid, sodium sulfate, sodium chloride, citric acid, dipotassium phosphate, potassium phosphate, sodium hydroxide, tocopherol, benzyl alcohol, benzoic acid and potassium sorbate and a carrier.

9. The window of opportunity regimen and composition of claim 1, wherein the first composition comprises three or more ingredients selected from the group consisting of glycerin, aqua, cetearyl alcohol, isocetyl alcohol, dimethicone, cetyl alcohol, Avena sativa kernel flour, Avena sativa kernel extract, Avena sativa kernel oil, caprylic/capric triglyceride, ceramide 3, ethylhexylglycerin, p-anisic acid, sodium cetearyl sulfate, palmitic acid, stearic acid, sodium sulfate, sodium chloride, citric acid, dipotassium phosphate, potassium phosphate, sodium hydroxide, tocopherol, benzyl alcohol, benzoic acid and potassium sorbate and a carrier.

10. The window of opportunity regimen and composition of claim 1, wherein the first composition comprises one or more of the following ingredients in the amounts set forth below:
a. caprylic/capric triglyceride, preferably from about 0.0% to about 1.0%, preferably from about 0.005% to about 1.0%;
b. citric acid, preferably from about 0.0% to about 0.1%, preferably from about 0.005% to about 0.1%;
c. benzoic acid, preferably from about 0.0% to about 2.0%, preferably from about 0.1% to about 2.0%;
d. potassium phosphate, preferably from about 0.0% to about 2.0%, preferably from about

0. 05% to about 2.0%;
e. dimethicone, preferably from about 0.0% to about 10.0%, preferably from about 1.0% to about 10.0%;
f. stearic acid; palmitic acid, preferably from about 0.0% to about 2.0%, preferably from about 0.1% to about 2.0%;
g. isocetyl alcohol, preferably from about 0.0% to about 10.0%, preferably from about 1.0% to about 10.0%;
h. Avena Sativa (Oat) Kernel Flour, preferably from about 0.0% to about 2.0%, preferably from about 0.1% to about 2.0%;
i. ceramide 3, preferably from about 0.0% to about 0.1%, preferably from about 0.005% to about 0.1%;
j. ethylhexylglycerin, preferably from about 0.0% to about 2.0%, preferably from about 0.1% to about 2.0%;
k. Avena Sativa (Oat) Kernel Oil, preferably from about 0.0% to about 1.0%, preferably from about 0.005% to about 1.0%;
l. Avena Sativa (Oat) Kernel Extract, which includes glycerin, potassium sorbate and water, preferably from about 0.0% to about 1.0%, preferably from about 0.005% to about 1.0%;
m. water, preferably from about 15.0% to about 40.0%, preferably from about 20.0% to about 40.0%;
n. glycerin, preferably from about 20.0% to about 50.0%, preferably from about 30.0% to about 50.0%;
o. sodium cetearyl sulfate, preferably from about 0.0% to about 2.0%, preferably from about 0.1% to about 2.0%;
p. dipotassium phosphate, preferably from about 0.0% to about 2.0%, preferably from about 0.1% to about 2.0%;
q. sodium hydroxide, preferably from about 0.0% to about 1.0%, preferably from about 0.005% to about 1.0%;
r. cetyl alcohol, preferably from about 0.0% to about 10.0%, preferably from about 1.0% to about 10.0%;
s. cetearyl alcohol, preferably from about 0.0% to about 10.0%, preferably from about 1.0% to about 10.0%;
t. benzyl alcohol, preferably from about 0.0% to about 2.0%, preferably from about 0.1% to about 2.0%;
u. p-anisic acid, preferably from about 0.0% to about 2.0%, preferably from about 0.1% to about 2.0%.

11. The window of opportunity regimen and composition of claim 1, wherein the first composition is formulated in a dosage form selected from a balm, a cream, a lotion, an emulsion, a serum, an ointment, a paste, intended for topical application to skin.

12. A window of opportunity kit comprising a first composition and instructions for use of the first composition in a regimen for preventing the onset of or treating atopic dermatitis in a subject.
